(19) United States
(12) Patent Application Publication
(10) Pub. No.: US 2013/0025597 A1
(43) Pub. Date: Jan. 31, 2013

(54) METHODS AND SYSTEMS FOR MONITORING A VENTILATED PATIENT WITH AN OXIMETER

(71) Applicants: Peter Doyle, Vista, CA (US); Mehdi Jafari, Laguna Hills, CA (US)

(72) Inventors: Peter Doyle, Vista, CA (US); Mehdi Jafari, Laguna Hills, CA (US)

(73) Assignee: Nellcor Puritan Bennett LLC, Boulder, CO (US)

(21) Appl. No.: 13/193,758

(22) Filed: Jul. 29, 2011

(51) Int. Cl. A61M 16/00

(52) U.S. Cl. 128/204.23

(57) ABSTRACT

This disclosure describes systems and methods for monitoring the ventilation of a patient being ventilated by a medical ventilator. The disclosure describes a novel approach for triggering the delivery of a breath to account for cardiogenic artifacts. The disclosure further describes a novel approach for calculating exhaled spirometry to eliminate the effect of cardiogenic artifacts.

Medical ventilator systems have been long used to provide supplemental oxygen support to patients. These ventilators typically comprise a source of pressurized oxygen which is fluidly connected to the patient through a conduit. Some ventilator systems monitor the patient during ventilation. In some systems, cardiogenic activity, or activity that is the result of the heart, is monitored through the use of a device such as an oximeter.

Further, medical ventilators may determine when a patient takes a breath in order to synchronize the operation of the ventilator with the natural breathing of the patient. In some instances, detection of the onset of inhalation and/or exhalation may be used to trigger one or more actions on the part of the ventilator. Accurate and timely measurement of patient airway pressure and lung flow in medical ventilators are directly related to maintaining patient-ventilator synchrony and spirometry calculations and pressure-flow-volume visualizations for clinical decision making. However, the beating of a patient's heart can affect the flow and/or pressure measurement. The heart's affect on the measured flow and/or pressure signals can have enough of an impact to exceed a triggering threshold, the result of which is the delivery of a breath at an inappropriate time.

### Monitoring a Ventilated Patient with an Oximeter

This disclosure describes systems and methods for monitoring the ventilation of a patient being ventilated by a medical ventilator. The disclosure describes a novel approach for triggering the delivery of a breath while accounting for cardiogenic artifacts. The disclosure further describes a novel approach for calculating exhaled spirometry while eliminating the effect of cardiogenic artifacts.

In part, this disclosure describes a method for monitoring the ventilation of a patient being ventilated by a medical ventilator system. The method includes:

a) monitoring at least one respiratory parameter to determine at least one monitored respiratory parameter of a patient being ventilated by a medical ventilator system;

b) monitoring a pulse rate of the patient with a pulse rate monitoring device;

c) determining one or more potential cardiogenic artifacts in the at least one monitored respiratory parameter;

d) correlating the potential cardiogenic artifacts with the pulse rate of the patient to verify cardiogenic artifacts in the at least one monitored respiratory parameter; and

e) removing the verified cardiogenic artifacts from the at least one monitored respiratory parameter to provide at least one adjusted respiratory parameter.

Yet another aspect of this disclosure describes a medical ventilator including:

a) a pneumatic gas delivery system, the pneumatic gas delivery system adapted to control a flow of gas from a gas supply to a patient via a ventilator breathing circuit;

b) a pulse rate monitoring device, the pulse rate monitoring device monitors a pulse rate of the patient;

c) at least one sensor, the at least one sensor monitors at least one respiratory parameter to provide at least one monitored respiratory parameter;

d) a correlation module, the correlation module is adapted to identify one or more potential cardiogenic artifacts in the at least one monitored respiratory parameter, and to correlate the potential cardiogenic artifacts with the pulse rate of the patient to verify cardiogenic artifacts in the at least one monitored respiratory parameter; and

e) a processor in communication with the pneumatic gas delivery system, the pulse rate monitoring device, the at least one sensor, and the correlation module.

The disclosure further describes a computer-readable medium having computer-executable instructions for performing a method for monitoring the ventilation of a patient being ventilated by a medical ventilator system. The method includes:

a) repeatedly monitoring at least one respiratory parameter to determine at least one monitored respiratory parameter of a patient being ventilated by a medical ventilator system;

b) repeatedly monitoring a pulse rate of the patient with a pulse rate monitoring device;

c) repeatedly determining one or more potential cardiogenic artifacts in the at least one monitored respiratory parameter;

d) repeatedly correlating the potential cardiogenic artifacts with the pulse rate of the patient to verify cardiogenic artifacts in the at least one monitored respiratory parameter; and

e) repeatedly removing the verified cardiogenic artifacts from the at least one monitored respiratory parameter to provide at least one adjusted respiratory parameter.

The disclosure also describes a medical ventilator system, including means for monitoring at least one respiratory parameter to determine at least one monitored respiratory parameter of a patient being ventilated by a medical ventilator system, means for monitoring a pulse rate of the patient with a pulse rate monitoring device, means for determining one or more potential cardiogenic artifacts in the at least one monitored respiratory parameter, means for correlating the potential cardiogenic artifacts with the pulse rate of the patient to verify cardiogenic artifacts in the at least one monitored respiratory parameter, and means for removing the verified cardiogenic artifacts from the at least one monitored respiratory parameter to provide at least one adjusted respiratory parameter.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments, systems, and methods described below and are not meant to limit the scope of the invention in any manner, which scope shall be based on the claims appended hereto.

FIG. 1 illustrates an embodiment of a ventilator system connected to a human patient.

FIG. 2 illustrates an embodiment of a method for monitoring the ventilation of a patient being ventilated by a medical ventilator system.

FIG. 3 illustrates an embodiment of a method for triggering the ventilation of a patient being ventilated by a medical ventilator system.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. The reader will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to control percentage of oxygen in the gas supplied by the ventilator to the patient, as well as triggering conditions for delivery of a breath. Further, it is desirable to monitor oxygen saturation level of the blood (SpO₂ level), patient airway pressure, lung flow, and heart rate of a patient. Accordingly, medical ventilator systems may be combined with an oximeter for non-invasively measuring the blood oxygen level (SpO2) and the heart rate of a patient.

On occasion, the expansion or contraction of the heart of a patient (herein known as a pulsatile event), such as a heartbeat, can interact with the lungs of the patient. Because the physical location of the heart is adjacent to the lungs, movements of the heart have the potential to touch and press against a lobe of the lungs and can cause enough volume change in the thorax of a patient to be interpreted as small respiratory flow or pressure changes by a sensor. The physical interaction of the heart with the lungs may be recorded by sensors for monitoring lung parameters such as, but not limited to, the flow, pressure, CO₂, and O₂ concentrations. The heart's impact on the monitored lung parameters are referred to herein as cardiogenic artifacts.

Flow and pressure are often used in calculating exhaled spirometry and/or in determining a trigger for the delivery of a breath to a patient by the medical ventilator. Cardiogenic artifacts, if significant enough, cause an inaccurate calculation of exhaled spirometry, and/or false triggering. Accordingly, the medical ventilator system disclosed herein can determine when a cardiogenic artifact occurs, and account for the presence of an artifact when calculating the exhaled spirometry or determining a triggering event.

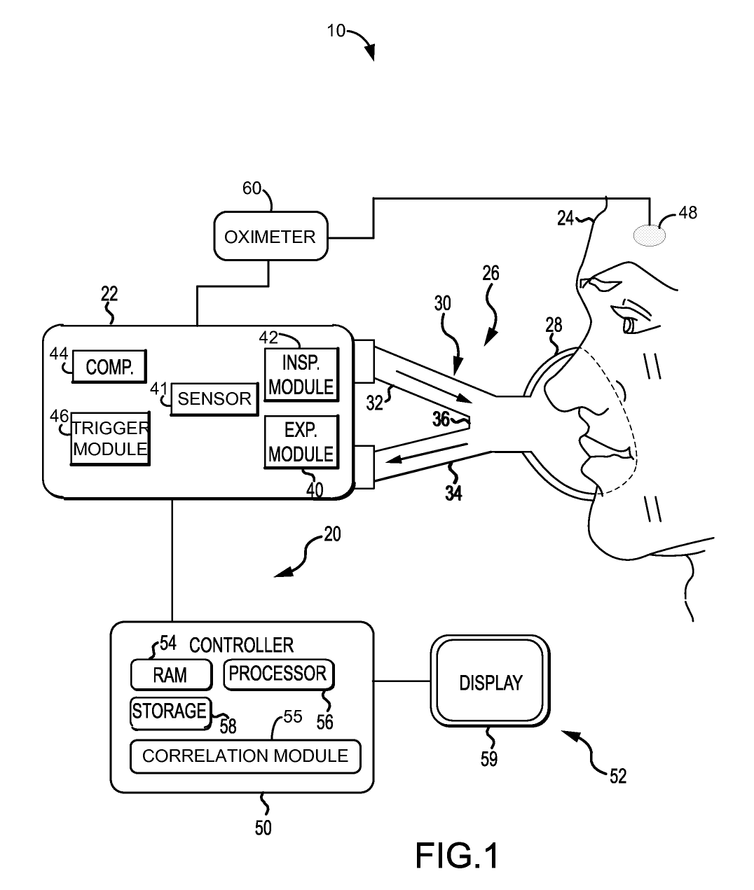

FIG. 1 illustrates an embodiment of a ventilator system 10 attached to a human patient 24. The ventilator system 10 includes a ventilator 20 in communication with a pulse rate monitoring device. The pulse rate monitoring device can be any device suitable for monitoring the pulse rate, heart rate, or heartbeat of a patient such as but not limited to an oximeter or an electrocardiogram (ECG). As used herein the terms “oximeter” and “ECG” are considered to be interchangeable in the present disclosure and in the claims and are used as non-limiting exemplary devices representative of a device suitable for monitoring the pulse rate, heart rate, or heartbeat of a patient. While “oximeter” and “ECG” refer to different devices, it is understood by a person of skill in the art that each may be used, as well as any other known or future devices for monitoring the pulse rate, heart rate, or heartbeat of a patient, for the purposes of this disclosure and for the purposes of the claims.

In one embodiment, as shown in FIG. 1, the pulse rate monitoring device is an oximeter 60. The oximeter 60, as shown in FIG. 1, is a completely separate and independent component from the ventilator 20. In an alternative embodiment, the oximeter 60 is located inside of the ventilator 20 and/or a pneumatic gas delivery system 22. As discussed above, the oximeter 60 and the ventilator 20 are in communication. This communication allows the ventilator 20 and the oximeter 60 to exchange data, commands, and/or instructions. In one embodiment, the oximeter 60 is in communication with a processor 56 of the ventilator 20.

The ventilator 20 includes the pneumatic gas delivery system 22 (also referred to as the pressure generating system 22) for circulating breathing gases to and from the patient 24 via a ventilation tubing system 26, which couples the patient 24 to the pneumatic gas delivery system 22 via a physical patient interface 28 and a ventilator breathing circuit 30.

The ventilator breathing circuit 30 could be a dual-limb or single-limb circuit 30 for carrying gas to and from the patient 24. In a dual-limb embodiment as shown, a wye fitting 36 may be provided as shown to couple the patient interface 28 to an inspiratory limb 32 and an expiratory limb 34 of the ventilator breathing circuit 30. Examples of suitable patient interfaces 28 include a nasal mask, nasal/oral mask (which is shown in FIG. 1), nasal prong, full-face mask, tracheal tube, endotracheal tube, nasal pillow, etc.

The pneumatic gas delivery system 22 may be configured in a variety of ways. In the present example, the pneumatic gas delivery system 22 includes an expiratory module 40 coupled with the expiratory limb 34 and an inspiratory module 42 coupled with the inspiratory limb 32. A compressor 44 or another source or sources of pressurized gas (e.g., pressured air and/or oxygen) is controlled by the pneumatic gas delivery system 22, such as by a gas regulator. The pneumatic gas delivery system 22 may include a variety of other components, including sources for pressurized air and/or oxygen, mixing modules, valves, sensors (e.g., sensor 41), tubing, filters, etc.

In one embodiment, the ventilator system 10 includes an oximeter sensor 48. The oximeter sensor 48 may be any sensor suitable for monitoring the blood oxygen level and pulse rate, heart rate, or heartbeat of the patient 24. As used herein the terms “pulse rate,” “heart rate,” and “heartbeat” are considered to be interchangeable in the present disclosure and in the claims. While “pulse rate,” “heartbeat,” and “heart rate” refer to different measurements, it is understood by a person of skill in the art that each may be used for the purposes of this disclosure and for the purposes of the claims. In one embodiment, the operator inputs the ideal body weight, gender, weight, and/or height of the patient 24, which inputs are used in combination with a pulse rate sensor to accurately calculate the pulse rate of a patient.

As illustrated in FIG. 1, the ventilator system 10 includes the oximeter 60. The oximeter 60 monitors the concentration of oxygen in the blood of the patient 24 (e.g., as SpO₂) from data gathered by the oximeter sensor 48. The oximeter 60 is in communication with the oximeter sensor 48. In one embodiment, the oximeter 60 monitors the pulse rate of the patient 24 with the oximeter sensor 48. For example, the oximeter 60 may monitor the pulse rate by monitoring the frequency of signal fluctuations caused by the expansion and contraction of the arterial blood vessels with each pulse as monitored by the oximeter sensor 48.

The oximeter 60 is in data communication with the ventilator 20. This data communication allows the ventilator 20 and the oximeter 60 to send data, instructions, and/or commands to each other. In one embodiment, the oximeter 60 is in communication with the processor 56 of the ventilator 20.

The ventilator system 10 includes one or more sensors 41 communicatively coupled to the ventilator 20 to monitor at least one respiratory parameter. In one embodiment, as illustrated in FIG. 1, the one or more sensors 41 are part of the pneumatic gas delivery system 22. According to embodiments, the one or more sensors 41 may communicate with various components of the ventilator 20, such as the inspiratory module 42, the expiratory module 40, a triggering module 46, a controller 50, and any other suitable components and/or modules. According to other embodiments, the one or more sensors 41 may be placed in any suitable location, e.g., within the ventilatory circuitry or other devices communicatively coupled to the ventilator. For example, the one or more sensors 41 may be affixed to the ventilatory tubing or may be imbedded in the tubing itself. The one or more sensors 41 may be coupled to the inspiratory 42 and/or expiratory modules 40 for detecting changes in circuit pressure and/or flow. Specifically, the one or more sensors 41 may include pressure transducers that detect changes in circuit pressure (e.g., electromechanical transducers including piezoelectric, variable capacitance, or strain gauge) or changes in a patient's muscular pressure. The one or more sensors 41 may further include various flowmeters for detecting airflow (e.g., differential pressure pneumotachometers). For example, some flowmeters may use obstructions to create a pressure decrease corresponding to the flow across the device (e.g., differential pressure pneumotachometers) and other flowmeters may use turbines such that flow may be determined based on the rate of turbine rotation (e.g., turbine flowmeters). Any sensory device useful for monitoring changes in measurable parameters during ventilatory treatment may be utilized in accordance with embodiments described herein. According to some embodiments, the one or more sensors 41 may be provided at or near the lungs (or diaphragm) for detecting a pressure in the lungs. Additionally or alternatively, the one or more sensors 41 may be affixed or embedded in or near the wye-fitting 36 and/or the patient interface 28.

According to some embodiments, the inspiration module 42 and/or the exhalation module 40 may be configured to synchronize ventilation with a spontaneously-breathing, or triggering, patient through the triggering module 46. The triggering module 46 is configured to detect changes in a monitored respiratory parameter and may initiate a transition from exhalation to inhalation (or from inhalation to exhalation) in response to said changes. Ventilators 20, depending on their mode of operation, may trigger automatically and/or in response to a detected change in a monitored respiratory parameter.

In some embodiments, the triggering module 46 of the ventilator 20 detects changes in a monitored respiratory parameter via the monitoring of a respiratory gas pressure, the monitoring of lung flow, direct or indirect measurement of nerve impulses, or any other suitable method for detecting changes in a monitored respiratory parameter. In embodiments where changes in a monitored respiratory parameter are detected by monitoring flow and/or pressure, the sensitivity of the ventilator 20 to changes in pressure and/or flow, also known as the triggering threshold, may be adjusted. For example, the higher the sensitivity of the triggering module 46 of the ventilator 20 to changes in pressure and/or flow (i.e., the lower the triggering threshold), the more sensitive the triggering module 46 is to patient triggering.

According to some embodiments, the triggering module 46 monitors the respiratory gas pressure in the breathing circuit 30 with the one or more sensors 41 to determine changes in a monitored respiratory parameter. The triggering module 46 detects a drop in pressure in the breathing circuit 30. The drop in circuit pressure detected by the triggering module 46 may indicate that the patient's respiratory muscles are creating a negative pressure gradient between the patient's lungs and the airway opening in an effort to inspire. The ventilator 20 interprets the drop in circuit pressure as patient inspiratory effort if it breaches the triggering threshold and initiates inspiration by delivering respiratory gases to the patient 24.

In other embodiments, the triggering module 46 of the ventilator 20 detects changes in lung flow in the breathing circuit 30 with the sensors 41 to determine changes in a monitored respiratory parameter. If the triggering module 46 of the ventilator 20 detects an increase in flow entering the lung (a drop in base flow monitored through the exhalation module) during exhalation that breaches the triggering threshold, this indicates to the triggering module 46 that the patient is attempting to inspire. In this case, the ventilator is detecting a drop in bias flow (or baseline flow) attributable to a redirection of gases into the patient's lungs (in response to a negative pressure gradient as discussed above). Bias flow refers to a constant flow existing in the breathing circuit 30 during exhalation that enables the ventilator to detect expiratory flow changes and patient triggering. For example, after the completion of a patient's active exhalation, while gases (constant bias flow) are generally flowing out of the ventilator during exhalation, a drop in flow monitored at the exhalation module may occur as some gas in the breathing circuit 30 is redirected and flows into the lungs in response to the negative pressure gradient created between the patient's lungs and the body's surface (connection point of patient to the tubing circuit). Thus, when the triggering module 46 of the ventilator 20 detects a drop in flow through the exhalation module below the bias flow by at least the threshold amount (e.g., 2 L/min below bias flow), the triggering module 46 interprets the drop as a patient trigger. Based on a detected patient trigger, the triggering module 46 instructs the ventilator 20 to initiate inspiration by delivering respiratory gases to the patient 24.

In one embodiment, the one or more sensors 41 may measure a respiratory parameter such as but not limited to flow and pressure. The monitored respiratory parameter can be used by the ventilator 20 and/or triggering module 46 to determine when to trigger the delivery of a breath. However, the action of the cardiac muscle or the pumping of the heart of the patient 24 can cause enough volume change in the thorax of the patient 24 to be interpreted as inspiratory flow and/or pressure changes caused by a respiratory effort by the one or more sensors 41. If the movement of the thorax is great enough, the movement of the thorax can lead to false exhaled spirometry readings and/or lead to the triggering module 46 interpreting the changes in flow and/or pressure as a trigger causing an unwanted delivery or cycling of a breath, herein known as a false trigger. Further, if the cardiogenic artifacts are large enough to be recorded by the one or more sensors 41, the cardiogenic artifacts are exhibited or recorded as brief, periodic, low-amplitude oscillatory disturbances in the monitored respiratory parameters. Typically, the larger a patient's heart, the larger the cardiogenic artifacts.

However, low-amplitude oscillatory disturbances found in the monitored respiratory parameters should be within a predetermined threshold to have been caused by the volume changes caused by the pumping of the patient's cardiac muscle. For example, in one embodiment, if the cardiogenic artifacts or oscillations registering in the monitored respiratory parameter of an adult patient are above about 0.7 Hertz, the oscillations are likely from the volume changes caused by the pumping of the patient's cardiac muscle and are referred to herein as potential cardiogenic artifacts. Further, in this or another embodiment, if the oscillations registering in the monitored respiratory parameter have a frequency of less than 0.7 Hertz, the oscillations should not be reasonably ascribed to volume changes caused by the pumping of the patient's cardiac muscle alone and generally should not be attributed to potential cardiogenic artifacts without additional information.

The controller 50 is in communication with the pneumatic gas delivery system 22, the oximeter 60, a display 59, and an operator interface 52, which may be provided to enable an operator to interact with the ventilator 20 (e.g., change ventilator settings, select operational modes, view monitored parameters, etc.). The controller 50 may include memory 54, one or more processors 56, storage 58, and/or other components of the type commonly found in command and control computing devices.

The memory 54 is non-transitory computer-readable storage media that stores software that is executed by the processor 56 and which controls the operation of the ventilator 20. In an embodiment, the memory 54 comprises one or more solid-state storage devices such as flash memory chips. In an alternative embodiment, the memory 54 may be mass storage connected to the processor 56 through a mass storage controller (not shown) and a communications bus (not shown). Although the description of non-transitory computer-readable media contained herein refers to a solid-state storage, it should be appreciated by those skilled in the art that non-transitory computer-readable storage media can be any available media that can be accessed by the processor 56. Non-transitory computer-readable storage media includes volatile and non-volatile, removable and non-removable media implemented in any method or technology for storage of information such as non-transitory computer-readable instructions, data structures, program modules or other data. Non-transitory computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 56.

In one embodiment, as illustrated in FIG. 1, the controller 50 further includes a correlation module 55. In another embodiment, not shown, the correlation module 55 is a separate component from or independent of the controller 50. In another embodiment, not shown, the correlation module 55 is a separate component from or independent of the ventilator 20. The correlation module 55 is in communication with other components of the ventilator 20 such as but not limited to the processor 56, the triggering module 46, the one or more sensors 41, the inspiratory module 42, the expiratory module 40, the oximeter 60, and the compressor 44.

In one embodiment, the correlation module 55 identifies potential cardiogenic artifacts and attempts to correlate the potential cardiogenic artifacts with the pulse rate of the patient 24. If the correlation module 55 determines that the pulse rate of the patient 24 correlates with potential cardiogenic artifacts, the correlation module 55 designates these potential cardiogenic artifacts as verified cardiogenic artifacts.

In one embodiment, the correlation module 55 removes or minimizes the distortions to the monitored respiratory parameter caused by verified cardiogenic artifacts by attempting to redraw or reconstruct the corrupted segment(s) of the monitored parameter as if there had been no cardiogenic artifacts in the first place. A respiratory parameter that has had the verified cardiogenic artifacts removed or minimized is referred to herein as an adjusted respiratory parameter. The adjusted respiratory parameter can be utilized by the correlation module 55, the controller 50, the oximeter 60, and/or the pneumatic gas delivery system 22 to calculate exhaled spirometry. Exhaled spirometry is useful in diagnosing asthma, chronic obstructive pulmonary disease, as well as certain other conditions that affect breathing. Exhaled spirometry is also used to check how well a patient's lungs are working during treatment. Further, exhaled spirometry can be used to measure respiratory parameters such as but not limited to tidal volume, forced expiratory flow, forced vital capacity, maximum voluntary ventilation, and peak expiratory flow. Exhaled spirometry can be calculated using the volume and/or flow of respiratory gas inhaled and/or exhaled by the patient 24. Calculating exhaled spirometry based on the adjusted respiratory parameter results in an accurate measurement. In some embodiments, the adjusted respiratory parameter can be used by the triggering module 46 to determine when to trigger the delivery of a breath.

In other embodiments, the correlation module 55 determines the timing of the verified cardiogenic artifacts and increases the triggering threshold utilized by the triggering module 46 at these determined times. The triggering threshold is increased enough to prevent a monitored respiratory parameter at the timing of a verified cardiogenic artifact from breaching the triggering threshold resulting in a false trigger, without sacrificing triggering sensitivity for other time periods. In other embodiments, the controller 50, the processor 56, the pneumatic system 22, and/or the ventilator 20 determine the timing and/or adjust the triggering threshold. It is appreciated by those skilled in the art that the triggering threshold can be increased, decreased, or changed in any suitable manner for ventilating a patient with a ventilator system 10. For example, if the triggering module 46 is utilizing a convention of a negative flow value as a triggering threshold, the triggering threshold may be decreased to a greater negative value during the determined times to account for the verified cardiogenic artifacts. For example, in some embodiments, the triggering threshold may vary from 1 to 4 L/min.

In some embodiments, the correlation module 55 determines that the pulse rate of the patient 24 does not correlate with potential cardiogenic artifacts. In these embodiments, the correlation module 55 does not remove or adjust the monitored respiratory parameter data. In other embodiments, if the correlation module 55 determines that the pulse rate of the patient 24 does not correlate with potential cardiogenic artifacts, the correlation module 55, the controller 50, the processor 56, the pneumatic system 22, the triggering module 46, and/or the ventilator 20 does not adjust the triggering threshold and/or adjusts the triggering threshold to a level suitable for use without cardiogenic artifacts.

Accordingly, the ventilator system 10 as described herein verifies the likelihood that small fluctuations or distortions found in the monitored respiratory parameters are coincident with the pulse rate of the patient 24, allowing the operator and/or the ventilator 20 to ignore those cardiogenic artifacts for the purpose of triggering breath delivery and/or calculating exhaled spirometry. In one embodiment, the correlation module 55 identifies potential cardiogenic artifacts, correlates the potential cardiogenic artifacts, and verifies the cardiogenic artifacts simultaneously or at the same time. In some embodiments, the correlation module 55 performs these functions in real-time or quasi-real-time. In other embodiments, the correlation module 55 performs these functions in some other type of sequential order.

In one embodiment, the correlation module 55 is activated upon user command. In an alternative embodiment, the correlation module 55 is activated based on a preset or a preselected ventilator setting. In another embodiment, the correlation module 55 is activated repeatedly based on a preset or a preselected ventilator setting. In yet another embodiment, the correlation module 55 is always active or activated based on data from the oximeter 60 and/or the ventilator 20.

In the depicted example, the operator interface 52 includes the display 59 that is touch-sensitive, enabling the display 59 to serve both as an input user interface and as an output device. In an alternative embodiment, the display 59 is not touch sensitive or an input user interface. The display 59 can display any type of ventilation information, such as sensor readings, parameters, commands, alarms, warnings, and/or smart prompts (i.e., ventilator-determined operator suggestions). The display 59 displays a monitored respiratory parameter to illustrate the interaction between the patient 24 being ventilated by the ventilator 20. In one embodiment, the display 59 displays a monitored respiratory parameter modified by the correlation module 55 to exclude verified cardiogenic artifacts. In alternative embodiments, upon operator selection or command, the display 59 displays a monitored respiratory parameter that was modified by the correlation module 55 to exclude verified cardiogenic artifacts with the removed cardiogenic artifacts reinserted. In some embodiments, the display 59 displays calculated exhaled spirometry.

In one embodiment, not shown, the oximeter 60 includes a display. In one embodiment, the oximeter display displays a pressure and/or flow wave-form modified by correlation module 55 to exclude verified cardiogenic artifacts. In other embodiments, upon operator selection, the oximeter display displays the pressure and/or flow wave-form modified by correlation module 55 so that both the pressure and/or flow wave-forms with and without the verified cardiogenic artifacts are shown. In some embodiments, the oximeter display displays calculated exhaled spirometry.

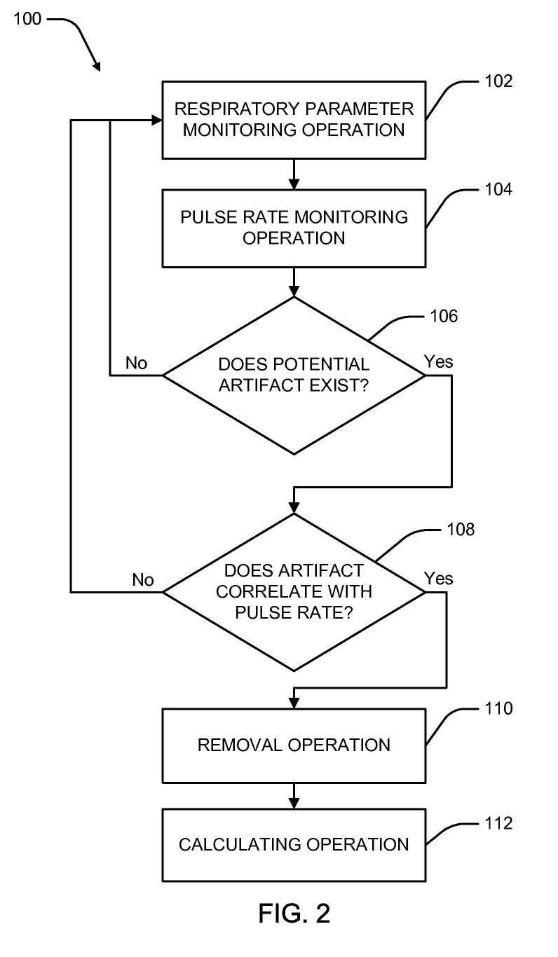

FIG. 2 illustrates an embodiment of a method 100 for monitoring a patient being ventilated by a medical ventilator system. As illustrated, the ventilator system during the method 100 performs a respiratory parameter monitoring operation 102. The ventilator system during the respiratory parameter monitoring operation 102 monitors at least one respiratory parameter of a patient such as, but not limited to, patient airway pressure and lung flow with at least one sensor. The at least one sensor is any sensor or combination of sensors suitable for monitoring a patient respiratory parameter. In one embodiment, the at least one sensor includes a flow sensor and/or a pressure sensor. In one embodiment, at least one of a monitored flow and a monitored pressure are detected by a plurality of sensors.

In some embodiments, the monitored respiratory parameter is utilized to determine an exhaled spirometry. In other embodiments, the monitored respiratory parameter is utilized to determine when to trigger a delivery of a breath to the patient. However, as noted above, the action of the cardiac muscle or the pumping of the heart of the ventilated patient can cause enough volume change in the thorax of the patient to be interpreted as a respiratory flow or pressure change by the at least one sensor. If the movement of the thorax is large enough, this movement can lead to inaccurate sensor readings and an inaccurate monitored respiratory parameter. The inaccurate monitored respiratory parameter can result in inaccurate exhaled spirometry calculations and/or lead to a false trigger. If the cardiogenic artifacts are large enough to affect the monitored respiratory parameter, the cardiogenic artifacts appear in the monitored respiratory parameter as brief, periodic, low-amplitude disturbances that disrupt an expected trace.

In addition to monitoring a respiratory parameter, the ventilator system during the method 100 performs a pulse rate monitoring operation 104. The ventilator system during the pulse rate monitoring operation 104 monitors a pulse rate, heartbeat, or heart rate of a patient being ventilated by a medical ventilator system with a pulse rate sensor. The pulse rate sensor is any sensor or combination of sensors suitable for monitoring the pulse rate or heart rate of the ventilated patient. In one embodiment, the pulse rate sensor is an oximeter sensor, an ECG sensor, a pulse rate sensor, or a cardiac monitor. In some embodiments, the pulse rate or heart rate of the ventilated patient is monitored using a monitor or sensor in combination with other data known to the ventilator, such as the ideal body weight, height, weight, and gender of the ventilated patient. In one embodiment, the ventilator system during the method 100 receives the ideal body weight, height, weight, and gender of the patient from operator input.

It is understood by a person of skill in the art that the pulse rate monitoring operation 104 and the respiratory parameter monitoring operation 102 may be performed in any order and/or simultaneously. In one embodiment, the pulse rate monitoring operation 104 and/or the respiratory parameter monitoring operation 102 are performed in real-time or quasi-real-time.

Next, the ventilator system during the method 100 performs a first decision operation 106. The ventilator system during the first decision operation 106 determines if there are any potential cardiogenic artifacts associated with the monitored respiratory parameter. Cardiogenic artifacts are recorded as small, periodic, low-level oscillations in the monitored respiratory parameter. Therefore, if periodic oscillations are present in the monitored respiratory parameter, the ventilator system during the first decision operation 106 determines that there are potential cardiogenic artifacts. Further, if periodic oscillations are not present in the monitored respiratory parameter, the ventilator system during the first decision operation 106 determines that there are not potential cardiogenic artifacts. Also, according to embodiments, an oscillation threshold may be established to limit a magnitude of the periodic oscillations that are reasonably caused by the heart. In some embodiments, the first decision operation 106 is performed via a correlation module, a controller, a processor, a pulse rate monitoring device, and/or a pneumatic system. If the ventilator system during the first decision operation 106 determines that the monitored respiratory parameter includes any potential cardiogenic artifacts, the ventilator system during the first decision operation 106 may perform a second decision operation 108. If the ventilator system during the first decision operation 106 determines that the monitored respiratory parameter does not include any potential cardiogenic artifacts, the ventilator system during the method 100 may return to the respiratory parameter monitoring operation 102.

In one embodiment, the first decision operation 106 is performed upon user or operator command. In an alternative embodiment, the first decision operation 106 is performed at a preset, preselected, and/or preconfigured time. In another embodiment, the first decision operation 106 is performed continuously and/or repeatedly based on a preset, a preconfigured, and/or a preselected time duration.

The ventilator system during the second decision operation 108 determines if the potential cardiogenic artifacts correlate with the monitored pulse rate of the ventilated patient. The ventilator system during the second decision operation 108 determines if small fluctuations or oscillations in the monitored respiratory parameter (i.e. the potential cardiogenic artifacts) are coincident with the pulse rate of a patient. Correlating potential cardiogenic artifacts with the monitored pulse rate of the patient confirms that the potential cardiogenic artifacts are actual recorded cardiogenic artifacts caused by the patient's heart. Accordingly, potential cardiogenic artifacts that correlate with the monitored pulse rate of the patient are referred to herein as verified cardiogenic artifacts. In some embodiments, the second decision operation 108 is performed with a correlation module, a controller, a processor, a pulse rate monitoring device, and/or a pneumatic system.

In one embodiment, the ventilator system during the second decision operation 108 determines at least one of a beginning, an intermediate point, and an end of a pulsatile event in the pulse rate. There may be a recording delay from when the ventilator system monitors a respiratory parameter to when the ventilator system records the monitored respiratory parameter. Accordingly, the timing of a cardiogenic artifact in the monitored respiratory parameter may not align with the timing of the pulsatile event. Therefore, the delay is calculated from the recorded pulsatile event to the timing of the recorded cardiogenic artifact in the monitored respiratory parameter by the ventilator system. For example, if the beginning of a pulsatile event is utilized, the delay until the beginning of the cardiogenic artifact is calculated. In one embodiment, the delay cannot be greater than a predetermined time value (e.g., 200 ms); otherwise the cardiogenic artifact should not reasonably be attributed to the pulsatile event. Once the delay is calculated by the ventilator system, a potential cardiogenic artifact can be verified if the potential cardiogenic artifact follows a pulsatile event by the predetermined delay.

If the ventilator system during the second decision operation 108 determines that the potential cardiogenic artifacts correlate with the monitored pulse rate or heart rate of the patient, the ventilator system during the second decision operation 108 may proceed to perform an artifact removal operation 110, and the potential cardiogenic artifacts are designated as verified cardiogenic artifacts. If the ventilator system during the second decision operation 108 determines that the potential cardiogenic artifacts do not correlate with the monitored pulse rate or heart rate of the patient, the ventilator system during the method 100 may return to perform the respiratory parameter monitoring operation 102.

In one embodiment, the second decision operation 108 is performed upon user or operator command. In an alternative embodiment, the second decision operation 108 is performed at a preset, preselected, and/or preconfigured time. In another embodiment, the second decision operation 108 is performed continuously and/or repeatedly based on a preset, a preconfigured, and/or a preselected time duration.

It is understood by a person of skill in the art that the first decision operation 106 and the second decision operation 108 may be performed simultaneously. In one embodiment, the first decision operation 106 and/or the second decision operation 108 are performed in real-time or quasi-real-time.

The ventilator system during the artifact removal operation 110 removes verified cardiogenic artifacts from the at least one monitored respiratory parameter to form at least one adjusted respiratory parameter. The artifact removal operation 110 may be performed by the correlation module, the controller, the processor, or any other suitable system for removing verified cardiogenic artifacts from a monitored respiratory parameter. In one embodiment, the ventilator system during the artifact removal operation 110 removes or minimizes the cardiogenic artifacts in the monitored respiratory parameter by attempting to redraw or reconstruct the recorded signal of the monitored parameter as if there had been no cardiogenic disruption in the first place, to form an adjusted respiratory parameter.

After the artifact removal operation 110, the ventilator system performs a calculating operation 112. In one embodiment, the ventilator system during the calculating operation 112 calculates an exhaled spirometry based on the adjusted respiratory parameter. In some embodiments, the exhaled spirometry is calculated using at least one of an adjusted flow and an adjusted pressure. In one embodiment, the ventilator system during the calculating operation 112 triggers the delivery of the breath based on the adjusted respiratory parameter breaching a triggering threshold. In one embodiment, the triggering threshold is at least one of a flow threshold, a pressure threshold, and/or a patient effort threshold. Utilizing the adjusted respiratory parameter for triggering can result in fewer false triggers than utilizing the monitored respiratory parameter for triggering the delivery of the breath to the patient.

In one embodiment, the ventilator system during the method 100 performs a display operation, not shown. In some embodiments, the ventilator system during the display operation displays an adjusted respiratory parameter wherein the verified cardiogenic artifacts have been removed. The display operation may be performed by a ventilator display and/or by another display. In an additional embodiment, the ventilator system during the display operation displays the monitored respiratory parameter with verified cardiogenic artifacts upon operator selection. In an additional embodiment, the ventilator system during the display operation displays at least one of the exhaled spirometry and the triggering threshold.

In one embodiment, the method 100 is performed by the ventilator system 10 described above and illustrated in FIG. 1. In some embodiments, a computer-readable medium having computer-executable instructions for performing a method for monitoring the ventilation of a patient being ventilated by a medical ventilator system are disclosed. This method includes repeatedly performing the steps disclosed in the method 100.

In another embodiment, a medical ventilator system is disclosed. The medical ventilator system includes: means for monitoring at least one respiratory parameter to determine at least one monitored respiratory parameter of a patient being ventilated by a medical ventilator system; means for monitoring a pulse rate of the patient with a pulse rate monitoring device; means for determining one or more potential cardiogenic artifacts in the at least one monitored respiratory parameter; means for correlating the potential cardiogenic artifacts with the pulse rate of the patient to verify cardiogenic artifacts in the at least one monitored respiratory parameter; and means for removing the verified cardiogenic artifacts from the at least one monitored respiratory parameter to provide at least one adjusted respiratory parameter.

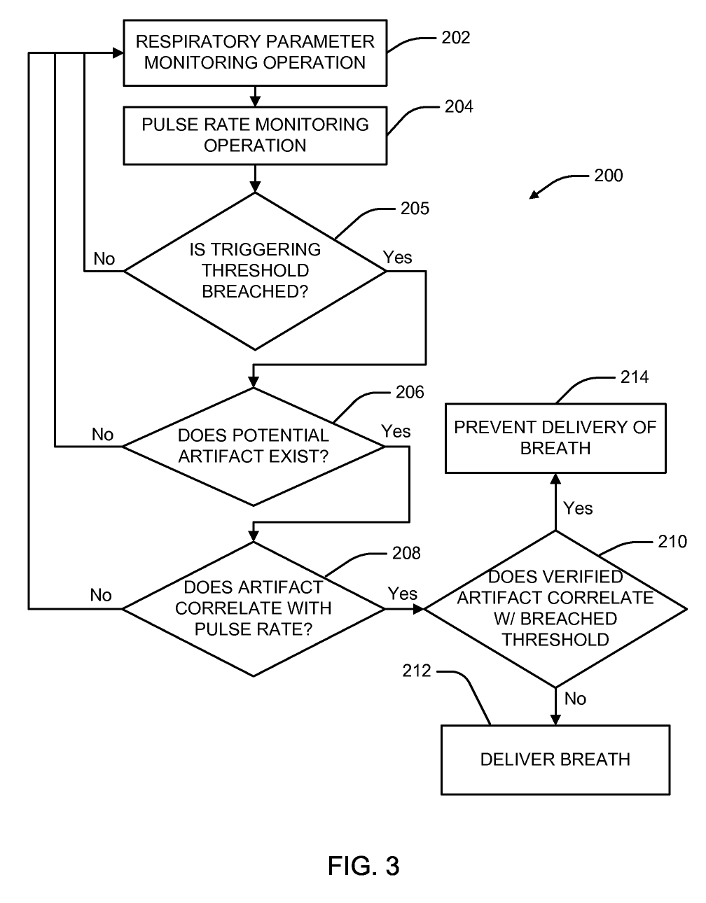

FIG. 3 illustrates an embodiment of a method 200 for triggering delivery of a breath to a patient being ventilated by a medical ventilator system. As illustrated, the ventilator system during the method 200 performs a respiratory parameter monitoring operation 202. The respiratory parameter monitoring operation 202 is the same operation as is described with respect to respiratory parameter monitoring operation 102 in the above method 100. The method 200 further includes a pulse rate monitoring operation 204, a first decision operation 206, and a second decision operation 208 which are the same as operations 104, 106, and 108, respectively, as described in the above method 100.

Additionally, the method 200 includes a threshold decision operation 205. The ventilator system during the threshold decision operation 205 determines if a triggering threshold is breached by at least one of the monitored respiratory parameters. In one embodiment, the triggering threshold is one of a flow threshold, a pressure threshold, and/or a patient effort threshold. If the ventilator system during the threshold decision operation 205 determines that the triggering threshold is not breached, the ventilator system during the method 200 returns to perform the respiratory parameter monitoring operation 202. Further, if the ventilator system during the threshold decision operation 205 determines that the triggering threshold is breached, the ventilator system performs the first decision operation 206. In one embodiment the threshold decision operation 205 is performed by the triggering module, the controller, the processor, the correlation module, the pneumatic gas delivery system, and/or any other suitable system for determining if the triggering threshold is exceeded by the monitored respiratory parameter.

It is understood by a person of skill in the art that the threshold decision operation 205 and the first decision operation 206 may be performed in any order and/or simultaneously. In one embodiment, the threshold decision operation 205 and/or the first decision operation 206 are performed in real-time. In one embodiment, the first decision operation 206 and a second decision operation 208 are performed before and/or simultaneously with the threshold decision operation 205. For example, if the ventilator system during the second decision operation 208 determines that the potential cardiogenic artifacts correlate with the monitored pulse rate of the patient, and the second decision operation 208 is performed before the threshold decision operation 205, the ventilator system during the second decision operation 208 selects to perform the threshold decision operation 205.

The method 200 includes the second decision operation 208. The second decision operation 208 is similar to the second decision operation as described in the above method 100. If the ventilator system during the second decision operation 208 determines that the potential cardiogenic artifacts do not correlate with the monitored pulse rate of the patient, the ventilator system during the method 200 returns to perform the respiratory monitoring operation 202. If the ventilator system during the second decision operation 208 determines that the potential cardiogenic artifacts correlate with the monitored pulse rate of the patient, the ventilator system during the second decision operation 108 proceeds to perform a trigger decision operation 210, and the potential cardiogenic artifacts are designated as verified cardiogenic artifacts.

Next, the method 200 includes the trigger decision operation 210. The ventilator system during the trigger decision operation 210 determines if the verified cardiogenic artifacts correlate with the breached trigger threshold, determined at threshold decision operation 205. The trigger decision operation 210 is responsible for deciding whether to trigger the delivery of a breath to the patient being ventilated. If the ventilator system during the trigger decision operation 210 determines that the verified cardiogenic artifact does not correlate with the breached trigger threshold, the ventilator system during the method 200 proceeds to a deliver breath operation 212. Alternately, if the ventilator system during the trigger decision operation 210 determines that the verified cardiogenic artifact correlates with the breached trigger threshold, the ventilator system during the method 200 performs a prevent breath operation 214. In one embodiment the trigger decision operation 210 is performed by the triggering module, the controller, the processor, the correlation module, the pneumatic gas delivery system, or any other suitable system for determining whether when the triggering threshold was exceeded by the monitored respiratory parameter correlates with the verified cardiogenic artifact.

The method 200 includes the deliver breath operation 212. The ventilator system during the deliver breath operation 212 delivers the breath to the patient. The ventilator system during the deliver breath operation 212 delivers few unwanted and/or unnecessary breaths since the ventilator system during the trigger decision operation 210 determined the breached triggering threshold was not a false trigger caused by cardiogenic artifacts. In one embodiment the ventilator system during the deliver breath operation 212 delivers the breath in any manner known, or any future delivery methods. In one embodiment the deliver breath operation 212 is performed by the triggering module, the controller, the processor, the correlation module, the pneumatic gas delivery system, or any other suitable system for delivering the breath.

The method 200 includes the prevent breath operation 214. The ventilator system during the prevent breath operation 214 prevents the delivery of a breath. In one embodiment, the ventilator system during the prevent breath operation 214 changes the triggering threshold because the ventilator system during the trigger decision operation 210 determined that the cardiogenic artifact correlated with the breached triggering threshold. In one embodiment, the threshold may be changed permanently, or for an extended period of time, or temporarily based on the timing of the cardiogenic artifact. For example, the ventilator system during the method 200 detects a pulsatile event and temporarily changes the triggering threshold the duration of the delay, as described in second decision operation 108 in the above method 100, after the pulsatile event, to account for the cardiogenic artifact. In one embodiment the prevent breath operation 205 is performed by the triggering module, the controller, the processor, the correlation module, the pneumatic gas delivery system, or any other suitable system for changing the triggering threshold.

In one embodiment, the ventilator system during the method 200 performs a display operation. The ventilator system during the display operation displays the monitored respiratory parameter wherein the verified cardiogenic artifacts have been removed. The display operation may be performed by a ventilator display and/or by an alternative display. In an additional embodiment, the ventilator system during the method 200 displays the monitored respiratory parameter with the verified cardiogenic artifacts reinserted upon operator selection. In some embodiments, the ventilator system during the display operation displays at least one of the triggering threshold and the changed triggering threshold.

In one embodiment, the method 200 is performed by the ventilator system illustrated in FIG. 1 and described above. In an alternative embodiment, a computer-readable medium having computer-executable instructions for performing a method for monitoring the ventilation of a patient being ventilated by a medical ventilator system are disclosed. This method includes repeatedly performing the steps disclosed in the method 200.

In another embodiment, a medical ventilator system is disclosed. The medical ventilator system includes: means for monitoring at least one respiratory parameter to determine at least one monitored respiratory parameter of a patient being ventilated by a ventilator system; means for determining that a triggering threshold has been breached for delivery of a breath based on the at least one monitored respiratory parameter; means for monitoring a pulse rate of the patient with a pulse rate monitoring device; means for determining one or more potential cardiogenic artifacts in the at least one monitored respiratory parameter; means for correlating the potential cardiogenic artifacts with the pulse rate of the patient to verify cardiogenic artifacts in the at least one monitored respiratory parameter; means for determining that the verified cardiogenic artifacts correlate with the step of determining that the triggering threshold has been breached; and means for preventing the delivery of the breath based on the step of determining that the verified cardiogenic artifacts correlate with the step of determining that the triggering threshold has been breached.

In an embodiment, the means for the medical ventilator system are illustrated in FIG. 1 and described in the above description of FIG. 1. However, the means for the ventilator system 10 described above and illustrated in FIG. 1 are exemplary only and are not meant to be limiting.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for monitoring the ventilation of a patient being ventilated by a medical ventilator system, the method comprising:
monitoring at least one respiratory parameter to determine at least one monitored respiratory parameter of a patient being ventilated by a medical ventilator system;
monitoring a pulse rate of the patient with a pulse rate monitoring device;
determining one or more potential cardiogenic artifacts in the at least one monitored respiratory parameter;
correlating the potential cardiogenic artifacts with the pulse rate of the patient to verify cardiogenic artifacts in the at least one monitored respiratory parameter; and
removing the verified cardiogenic artifacts from the at least one monitored respiratory parameter to provide at least one adjusted respiratory parameter.

2. The method of claim 1, further comprising:
determining exhaled spirometry based on the at least one adjusted respiratory parameters.

3. The method of claim 2, further comprising:
displaying at least one of the at least one adjusted respiratory parameter and the determined exhaled spirometry.

4. The method of claim 1, further comprising:
triggering a delivery of a breath based on the at least one adjusted respiratory parameter.

5. The method of claim 1, wherein the step of correlating the potential cardiogenic artifacts with the pulse rate comprises:
determining at least one of a beginning, an end, and an intermediate point of a pulsatile event in the pulse rate; and
calculating a delay in timing from at least one of the determined beginning, the determined intermediate point, and the determined end of the pulsatile event to a timing of the potential cardiogenic artifacts.

6. The method of claim 1, wherein the at least one respiratory parameter is one of flow and pressure.

7. The method of claim 1, wherein the pulse rate monitoring device is selected from a group of an oximeter and an ECG.

8. The method of claim 1, wherein at least one of an ideal body weight, a gender, a weight, and a height are utilized in combination with the pulse rate monitoring device to determine a pulse rate of the patient.

9. A method for triggering the ventilation of a patient being ventilated by a medical ventilator system, the method comprising:
monitoring at least one respiratory parameter to determine at least one monitored respiratory parameter of a patient being ventilated by a ventilator system;
determining that a triggering threshold has been breached for delivery of a breath based on the at least one monitored respiratory parameter;
monitoring a pulse rate of the patient with a pulse rate monitoring device;
determining one or more potential cardiogenic artifacts in the at least one monitored respiratory parameter;
correlating the potential cardiogenic artifacts with the pulse rate of the patient to verify cardiogenic artifacts in the at least one monitored respiratory parameter;
determining that the verified cardiogenic artifacts correlate with the step of determining that the triggering threshold has been breached; and
preventing the delivery of the breath based on the step of determining that the verified cardiogenic artifacts correlate with the step of determining that the triggering threshold has been breached.

10. The method of claim 9, wherein the at least one respiratory parameter is one of flow and pressure.

11. The method of claim 9, wherein the step of preventing the delivery of the breath comprises:
changing the triggering threshold.

12. The method of claim 9, wherein the triggering threshold is at least one of a flow threshold and a pressure threshold.

13. The method of claim 9, wherein the step of determining that the potential cardiogenic artifacts correlate with the pulse rate comprises:
determining at least one of a beginning, an end, and an intermediate point of a pulsatile event in the pulse rate; and
calculating a delay in timing from at least one of the determined beginning, the determined intermediate point, and the determined end of the pulsatile event to a timing of the potential cardiogenic artifacts.

14. The method of claim 13, wherein the step of preventing the delivery of the breath further comprises:
determining when to change the triggering threshold based on the delay and the pulse rate.

15. A medical ventilator system, comprising:
a pneumatic gas delivery system, the pneumatic gas delivery system adapted to control a flow of gas from a gas supply to a patient via a ventilator breathing circuit;
a pulse rate monitoring device, the pulse rate monitoring device monitors a pulse rate of the patient;
at least one sensor, the at least one sensor monitors at least one respiratory parameter to provide at least one monitored respiratory parameter;
a correlation module, the correlation module is adapted to identify one or more potential cardiogenic artifacts in the at least one monitored respiratory parameter, and to correlate the potential cardiogenic artifacts with the pulse rate of the patient to verify cardiogenic artifacts in the at least one monitored respiratory parameter; and
a processor in communication with the pneumatic gas delivery system, the pulse rate monitoring device, the at least one sensor, and the correlation module.

16. The medical ventilator system of claim 15, wherein the correlation module removes the verified cardiogenic artifacts from the at least one monitored respiratory parameter to provide at least one adjusted respiratory parameter.

17. The medical ventilator system of claim 16, further comprising:
a display in communication with the processor, the display displays at least one of the at least one monitored respiratory parameter and the at least one adjusted respiratory parameter.

18. The medical ventilator system of claim 16, wherein the correlation module determines exhaled spirometry based on the at least one adjusted respiratory parameter.

19. The medical ventilator system of claim 18, further comprising:
a display in communication with the processor, the display displays the determined exhaled spirometry.

20. The medical ventilator system of claim 16, further comprising:
a triggering module in communication with the processor, the triggering module is adapted to trigger a delivery of a breath to the patient based on the at least one adjusted respiratory parameter.

21. The medical ventilator system of claim 15, further comprising:
a triggering module in communication with the processor, the triggering module triggers a delivery of a breath to the patient based on the at least one monitored respiratory parameter exceeding a triggering threshold, wherein the triggering module is configured to adjust the triggering threshold to compensate for the verified cardiogenic artifacts in the at least one monitored respiratory parameter.

22. The medical ventilator system of claim 21, wherein the triggering module changes the triggering threshold periodically to compensate for the verified cardiogenic artifacts in the at least one monitored respiratory parameter based on a delay from the pulse rate to the verified cardiogenic artifact in the at least one monitored respiratory parameter.

23. A computer-readable medium having computer-executable instructions for performing a method for monitoring the ventilation of a patient being ventilated by a medical ventilator system, the method comprising:
repeatedly monitoring at least one respiratory parameter to determine at least one monitored respiratory parameter of a patient being ventilated by a medical ventilator system;
repeatedly monitoring a pulse rate of the patient with a pulse rate monitoring device;
repeatedly determining one or more potential cardiogenic artifacts in the at least one monitored respiratory parameter;
repeatedly correlating the potential cardiogenic artifacts with the pulse rate of the patient to verify cardiogenic artifacts in the at least one monitored respiratory parameter; and
repeatedly removing the verified cardiogenic artifacts from the at least one monitored respiratory parameter to provide at least one adjusted respiratory parameter.

24. A computer-readable medium having computer-executable instructions for performing a method for triggering the ventilation of a patient being ventilated by a medical ventilator system, the method comprising:
repeatedly monitoring at least one respiratory parameter to determine at least one monitored respiratory parameter of a patient being ventilated by a ventilator system;
repeatedly determining that a triggering threshold has been breached for delivery of a breath based on the at least one monitored respiratory parameter;
repeatedly monitoring a pulse rate of the patient with a pulse rate monitoring device;
repeatedly determining one or more potential cardiogenic artifacts in the at least one monitored respiratory parameter;
repeatedly correlating the potential cardiogenic artifacts with the pulse rate of the patient to verify cardiogenic artifacts in the at least one monitored respiratory parameter;
repeatedly determining that the verified cardiogenic artifacts correlate with the step of determining that the triggering threshold has been breached; and
repeatedly preventing the delivery of the breath based on the step of determining that the verified cardiogenic artifacts correlate with the step of determining that the triggering threshold has been breached.

25. A medical ventilator system, comprising:
means for monitoring at least one respiratory parameter to determine at least one monitored respiratory parameter of a patient being ventilated by a medical ventilator system;
means for monitoring a pulse rate of the patient with a pulse rate monitoring device;
means for determining one or more potential cardiogenic artifacts in the at least one monitored respiratory parameter;
means for correlating the potential cardiogenic artifacts with the pulse rate of the patient to verify cardiogenic artifacts in the at least one monitored respiratory parameter; and
means for removing the verified cardiogenic artifacts from the at least one monitored respiratory parameter to provide at least one adjusted respiratory parameter.

26. A medical ventilator system, comprising:
means for monitoring at least one respiratory parameter to determine at least one monitored respiratory parameter of a patient being ventilated by a ventilator system;
means for determining that a triggering threshold has been breached for delivery of a breath based on the at least one monitored respiratory parameter;
means for monitoring a pulse rate of the patient with a pulse rate monitoring device;
means for determining one or more potential cardiogenic artifacts in the at least one monitored respiratory parameter;
means for correlating the potential cardiogenic artifacts with the pulse rate of the patient to verify cardiogenic artifacts in the at least one monitored respiratory parameter;
means for determining that the verified cardiogenic artifacts correlate with the step of determining that the triggering threshold has been breached; and
means for preventing the delivery of the breath based on the step of determining that the verified cardiogenic artifacts correlate with the step of determining that the triggering threshold has been breached.
